(19) United States
(12) Patent Application Publication
(10) Pub. No.: US 2013/0006160 A1
(43) Pub. Date: Jan. 3, 2013

(54) Knee saving device

(76) Inventor: Jeffrey Cacace, East Northport, NY (US)

(21) Appl. No.: 13/135,271

(22) Filed: Jun. 30, 2011

(51) Int. Cl. A61F 5/00

(52) U.S. Cl. 602/26

(57) ABSTRACT

A device for supporting a wearer's knee prevents the knee from straightening during sleep. The device includes a sleeve adapted to be placed around a wearer's knee, and a rear pad connected to the sleeve. When the sleeve is placed around a person's knee, the pad causes the knee to be kept in an unlocked position when the person lies on their back. The rear pad has side walls that extend around the sleeve to surround the sides of the knee when the sleeve is placed around the knee. There is a front pad disposed opposite the rear pad on the sleeve. The front pad is positioned below the knee when the sleeve is placed around the knee, and prevents the knee from pressing on a surface when the wearer is lying face-down.

### BACKGROUND OF THE INVENTION

1. Field of the Invention

The present invention relates to a device for positioning a wearer's knee while sleeping. In particular, the invention relates to a sleeve that is fitted around a person's knee and keeps the knee in a bent position while sleeping.

2. The Prior Art

Knee pain is a common complaint among many adults, especially as they age. Obesity, overuse and inadequate footwear contribute to this problem. As the cartilage under the patella (kneecap) wears away, the bones rub against each other, which causes pain and discomfort. This is especially prevalent when the knee is in a straightened or locked position. This is the position where the bones are situated so as to rub and press against each other.

Thus, it would be desirable to prevent the knee from straightening unless such a position is required for movement. However, during sleep, it is impossible to control the position of one's legs.

### SUMMARY OF THE INVENTION

It is therefore an object of the invention to provide a device that prevents a person's leg from straightening during sleep. It is another object of the invention to provide such a device that is simple to put on and remove, and which is comfortable to wear while sleeping.

These and other objects are accomplished by a device for supporting a wearer's knee and preventing the knee from straightening during sleep. The device comprises a sleeve adapted to be placed around a wearer's knee, and a rear pad connected to the sleeve. When the sleeve is placed around a person's knee, the pad causes the knee to bend and be maintained in an unlocked position when the person lies on their back. This is because the bulk of the pad pushes the knee up into an unlocked position. The rear pad also has side walls that extend around the sleeve to surround the sides of the knee when the sleeve is placed around the knee. This keeps pressure off of the knee when the wearer of the device is sleeping on their side.

There is also preferably a front pad disposed opposite the rear pad on the sleeve. The front pad is positioned below the knee when the sleeve is placed around the knee, and is adapted to prevent the kneecap from pressing on a surface when the wearer is lying face-down. This is because the bulk of the front pad touches the sleeping surface, and relieves any pressure on the patella that would be caused by the sleeping surface.

The sleeve can have an aperture on the side opposite the rear pad for accommodating the patella when the sleeve is placed around the knee. This ensures that the sleeve is always properly positioned, as the patella extends through the aperture and prevents any sliding of the sleeve.

The sleeve is preferably formed from an elastic material, so that it can be used by people of varying sizes. The material is also preferably breathable, so that it will not be uncomfortable when worn for long periods of time. The sleeve can be a unitary construct, that can be slipped on the user's leg, or can be in the form of an open panel that can be wrapped around the user's leg and secured along a longitudinal seam. The sleeve could be secured via snaps, hooks, or a hook-and-eye type closure such as VELCRO®. With this embodiment, the sleeve could be worn over clothing or stockings, or on the bare skin, depending on the needs of the wearer.

In a preferred embodiment, the rear pad has a height of at least 5 inches in a middle region, directly behind the patella. This is sufficient elevation to relieve any pressure on the cartilage during use. The rear pad can have a height of at least 6 inches at the sides, so that the rear pad extends up and around the sides of the leg to cushion the sides of the leg with respect to each other.

The rear pad is preferably removable from the sleeve, so that the sleeve can be washed without damaging the pad. The rear pad is preferably formed from a foam material, but any soft material could be used.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a side view of the device according to the invention on a user's leg;

FIG. 2 shows a side view of the device with the user lying face-up;

FIG. 3 shows a cross-sectional view of the device according to lines III-III of FIG. 1; and

FIG. 4 shows a side view of another embodiment of the device with the user lying face-down.

### DETAILED DESCRIPTION OF THE PREFERRED EMBODIMENT

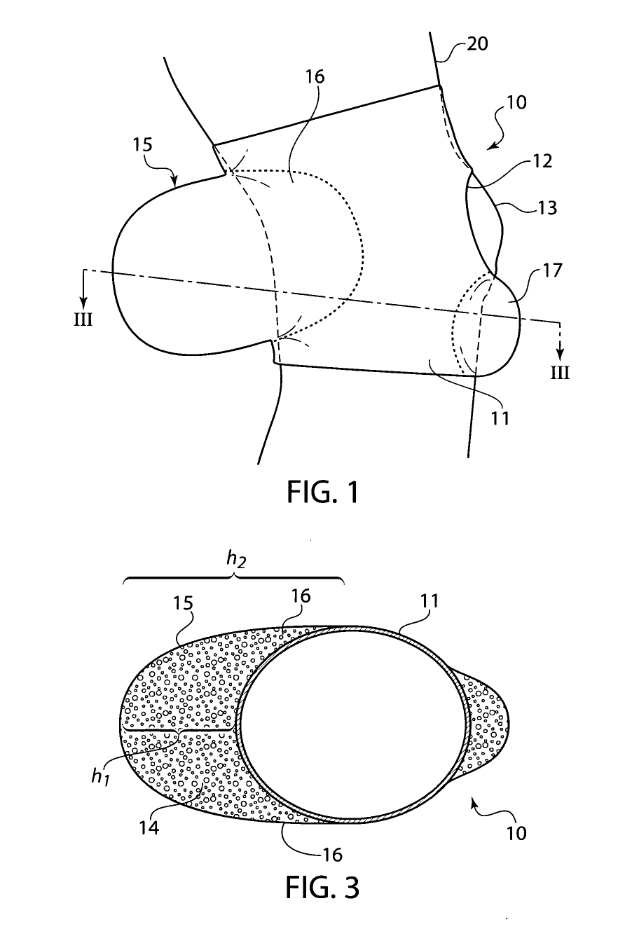

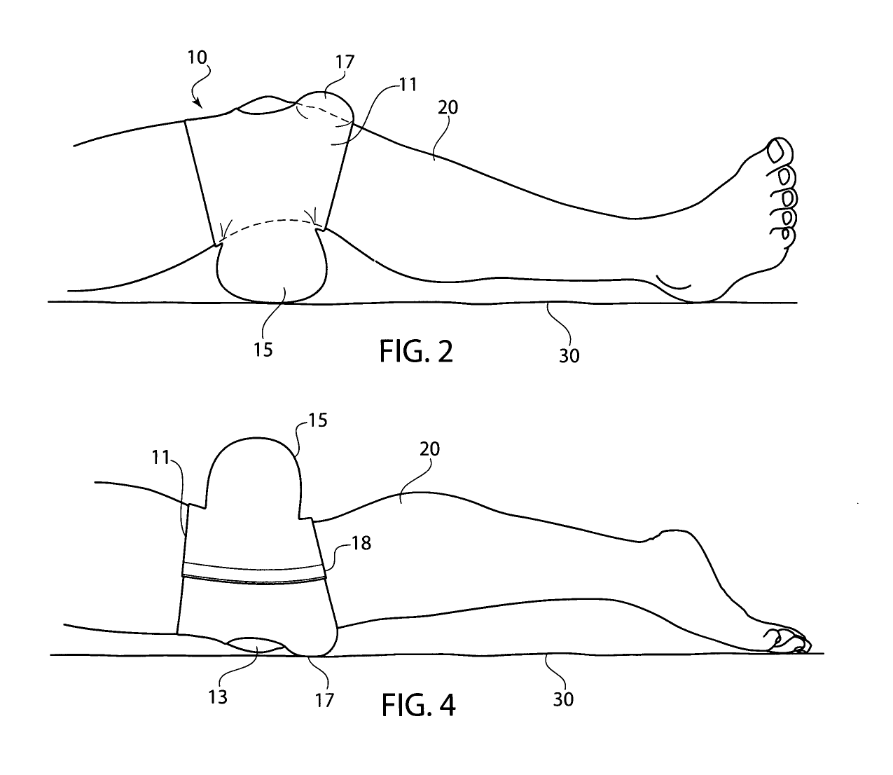

Referring now in detail to the drawings and, in particular, FIG. 1 shows a side view of the device 10 as positioned on a leg 20 of a person. Device 10 consists of a sleeve 11, with a cut-out 12 for the wearer's patella 13. Sleeve 11 can be constructed as a tube that is slid onto the user's leg as shown in FIG. 2, or can be a planar element that is secured along a seam, as shown in FIG. 4. A rear pad 15 is attached to the back of the sleeve 11, opposite the cut-out 12. Pad 15 has side sections 16 that extend around the sides of sleeve 11. As shown in FIG. 3, the interior 14 of pad 15 is preferably made of a foam material, but any soft material could be used. There is also a front pad 17 disposed just below cut-out 12 on the front of the device 10. Front pad 17 is smaller than rear pad 15.

As shown in FIG. 2, when the wearer lies down on a surface 30 facing upward, rear pad 15 forces the wearer's knee into an unlocked or bent position. This relieves stress and friction on the knee components, especially the cartilage and ligaments. Device 10 prevents the knee from bending through the wearer's sleep cycle, so that reduced knee pain results.

As shown in FIG. 4, when the person is lying face-down, front pad 17 raises the patella 13 slightly to relieve any pressure caused by surface 30. In addition, since rear pad 15 extends around the sides of sleeve 11, as shown in FIGS. 1 and 3, the pad prevents the person's knees from touching each other when the person is sleeping on their side.

As shown in FIG. 4, the sleeve 11 can be constructed so that it can be wrapped around the wearer's leg 20 and secured along a seam 18. This allows the device 10 to be worn over stockings or articles of clothing if desired. Seam 18 could be secured via snaps, hooks, hook-and-eye type closures such as VELCRO®, or any other desired manner.

The rear pad has a height h₁ of at least 5 inches in a middle region, directly behind the patella. This is sufficient elevation to relieve any pressure on the cartilage during use. The rear pad can have a height h₂ of at least 6 inches at the sides, so that the rear pad extends up and around the sides of the leg to cushion the sides of the leg with respect to each other.

Thus, the device according to the invention cushions and relieves stress regardless of the sleeping position of the wearer. The rear pad 15 keeps the knee in an unlocked position, while the front pad prevents pressure on the patella, and the side extensions of rear pad 15 prevent the knees from rubbing against each other.

Accordingly, while only single embodiment of the present invention has been shown and described, it is obvious that many changes and modifications may be made thereunto without departing from the spirit and scope of the invention.

What is claimed is:

1. A device for supporting a knee of a person, comprising:
a sleeve adapted to be placed around the knee; and
a rear pad connected to the sleeve, the rear pad being connected to the sleeve and preventing the knee from straightening when the sleeve is placed around the knee and the person is lying on their back, the rear pad having side walls that extend around the sleeve to surround the sides of the knee when the sleeve is placed around the knee.

2. The device according to claim 1, further comprising a front pad disposed opposite the rear pad on the sleeve, said front pad being positioned below the knee when the sleeve is placed around the knee, said front pad being adapted to prevent the knee from pressing on a surface when the wearer is lying face-down.

3. The device according to claim 1, wherein the sleeve has an aperture therein on a side opposite the rear pad, said aperture being adapted for accommodating the patella when the sleeve is placed around the knee.

4. The device according to claim 1, wherein the sleeve is formed from an elastic material.

5. The device according to claim 1, wherein the rear pad has a height of at least 5 inches in a middle region thereof.

6. The device according to claim 1, wherein the rear pad has a height of at least 6 inches at side regions thereof.

7. The device according to claim 1, wherein the rear pad is removable from the sleeve.

8. The device according to claim 1, wherein the rear pad is formed from a foam material.

9. The device according to claim 1, wherein the sleeve is constructed as a tube that is slid onto a wearer's leg.

10. The device according to claim 1, wherein the sleeve is constructed as a planar element that is wrapped around the wearer's leg and secured along a seam.
